(19) United States
(12) Patent Application Publication
(10) Pub. No.: US 2024/0277324 A1
(43) Pub. Date: Aug. 22, 2024

(54) SEAL ASSEMBLY AND CONTROL STRUCTURE FOR FLEXIBLE ELOGNATE DEVICE

(71) Applicant: INTUITIVE SURGICAL OPERATIONS, INC., Sunnyvale, CA (US)

(72) Inventors: Pak Yan Yuen, Los Altos, CA (US); Joseph Bogusky, San Jose, CA (US); Joseph Callol, San Francisco, CA (US); Jason Chan, Fremont, CA (US); Vijay Thadani, San Jose, CA (US); Arnold Chen, San Jose, CA (US); Saul Hernandez Morales, San Jose, CA (US); Worth Walters, Campbell, CA (US); David Solum, Santa Cruz, CA (US); Wadiyat Abbas, Cupertino, CA (US); Majeed Foad, Sunnyvale, CA (US); Lucas Gordon, Santa Barbara, CA (US); Stephen Kaplan, San Carlos, CA (US)

(21) Appl. No.: 18/442,897

(22) Filed: Feb. 15, 2024

Related U.S. Application Data
(60) Provisional application No. 63/485,310, filed on Feb. 16, 2023.

(51) Int. Cl. A61B 17/00; A61B 34/00; A61B 34/30; A61B 34/37

(52) U.S. Cl. CPC A61B 17/00234; A61B 34/37; A61B 34/71; A61B 2017/00314; A61B 2017/00327; A61B 2034/301; A61B 2034/303

(57) ABSTRACT

Flexible elongate devices and methods include an articulable body portion and a control structure attached with one or more pull wires that control articulation of the articulable body portion. The control structure includes a metallic material that defines a sealing surface functionalized by a primer. A polymer material disposed onto the sealing surface of the control structure creates a seal between the control structure and polymer material. Flexible elongate devices and methods also include a control structure and a pull wire configured to control articulation of the flexible elongate device. The control structure includes a plurality of control segments stacked along a longitudinal axis, where each of the control segments define a pull wire aperture, where pull wire apertures of adjacent control segments are offset relative to one another, such that a portion of the pull wire extending through the pull wire apertures has a non-linear shape.

### CROSS-REFERENCE TO RELATED APPLICATION

This application claims the priority benefit of U.S. Provisional Application No. 63/485,310, filed Feb. 16, 2024, which is hereby incorporated by reference herein in its entirety.

### FIELD

Disclosed embodiments relate to assemblies for a flexible elongate device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

According to some embodiments, a flexible elongate device includes an articulable body portion and a control structure attached with one or more pull wires that control articulation of the articulable body portion. The control structure includes a metallic material that defines a sealing surface functionalized by a primer. The flexible elongate device further includes a polymer material disposed onto the sealing surface of the control structure to create a seal between the control structure and polymer material.

According to some embodiments, a method for assembly of a flexible elongate device includes functionalizing a metallic sealing surface of a control structure with a primer to create a functionalized sealing surface, attaching one or more pull wires that control articulation of an articulable portion of the flexible elongate device to the control structure, and disposing a polymer material onto the functionalized sealing surface to create a seal between the control structure and the polymer material.

According to some embodiments, a flexible elongate device includes a control structure and a pull wire configured to control articulation of the flexible elongate device. The control structure includes a plurality of control segments stacked along a longitudinal axis thereof, where each of the control segments define a tool channel aperture and a pull wire aperture. The tool channel apertures of the plurality of control segments are coaxially aligned to at least partially form a tool channel of the flexible elongate device and the pull wire apertures of adjacent control segments are offset relative to one another in a direction orthogonal to the longitudinal axis of the control structure. The pull wire extends through the pull wire apertures of the plurality of control segments, such that a portion of the pull wire extending through the pull wire apertures has a non-linear shape.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4 is a simplified diagram of a flexible elongate device according to some embodiments.

FIG. 5 is a simplified diagram of a distal section of a flexible elongate device according to some embodiments.

FIGS. 6 and 7 are simplified diagrams of a control structure assembly of a flexible elongate device according to some embodiments.

FIG. 8 is a simplified diagram of a flexible elongate device according to some embodiments.

FIG. 9 is a simplified diagram of a flexible elongate device according to some embodiments.

FIG. 10 is a flowchart for a method of assembly of a flexible elongate device according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

In some examples, flexible and/or steerable elongate devices can include assemblies requiring sealing between a polymer material and a metallic material. These seals can be important to prevent the entrapment of biological matter and chemicals. Further, these seals should be able to withstand the various environmental and chemical exposures, as well as mechanical loading imparted thereto, through the lifespan of the flexible and/or steerable elongate device. Accordingly, it would be advantageous to provide devices and methods having strong seals formed between polymer and metallic materials to ensure proper function and life span for flexible and/or steerable elongate devices.

The disclosure relates to functionalizing a seal surface of a metallic control structure of a flexible elongate device for sealing with a polymer material and a flexible elongate device having a seal formed between a polymer material and a functionalized sealing surface of a metallic control structure. For example, this disclosure is suitable for creating a seal between an outer jacket and/or an inner liner and a metallic control structure of a flexible elongate device. A phosphonate or a silane, such as aminosilane, or other primer is applied to the metallic control structure to functionalize a sealing surface thereon. The molecules of the primer react with the metallic sealing surface to become covalently bonded with the metal. As a result, after priming, the functionalized sealing surface is covered by the reacted primer molecules. In some instances, the control structure can include a plurality of functionalized sealing surfaces, including longitudinal and/or transverse sealing surfaces.

The functionalization of the sealing surface(s) of the control structure enhances the adhesion of the sealing surface with polymer materials, such as polyurethane, and therefore creates a strong seal between the two materials preventing the entrapment of biological matter and chemicals between the materials.

The disclosure also relates to a flexible elongate device having a control structure assembly with a circuitous internal pull wire path that secures the pull wire within control structure segments. The control structure assembly can include one or more pull wires, including one, two, or four pull wires, to impart a desired degree of control over articulation of the flexible elongate device. For each pull wire, the plurality of control structure segments include a pull wire opening extending axially therethrough. The plurality of control structure segments are stacked along a longitudinal axis of the control structure and the pull wire openings of adjacent control structure segments are offset in a direction orthogonal, e.g., circumferentially and/or radially, relative to one another.

The non-linear shape of the secured portion of the pull wire advantageously secures the pull wire to the control structure without the use of crimp ferrules, laser welding and the required assemblies associated with laser welding, soldering, or other securing techniques. The control structure assembly can also be configured to be suitable for pull wires like fiber, polymer, and cable pull wires, that are flexible and very strong in tension, but are problematic to secure to the control structure, as knotting or similar techniques can weaken the material.

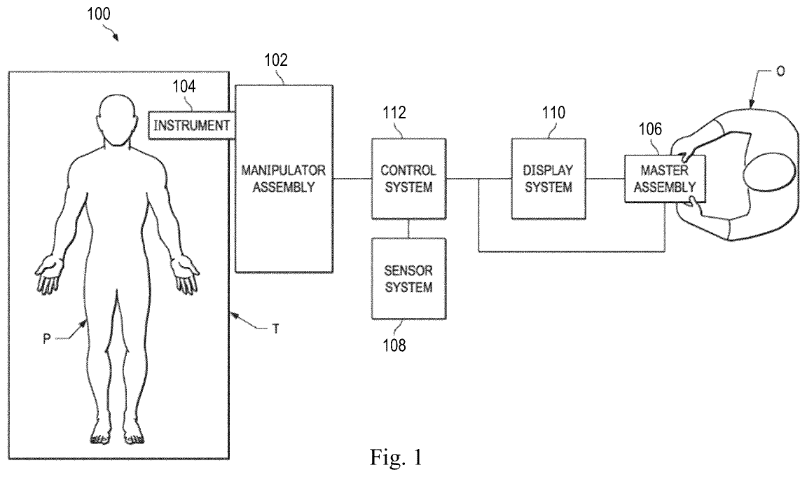

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

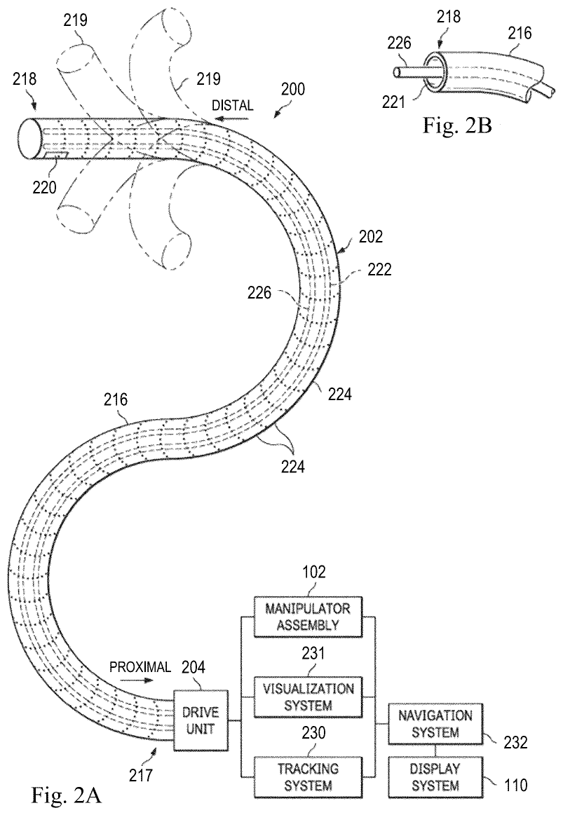

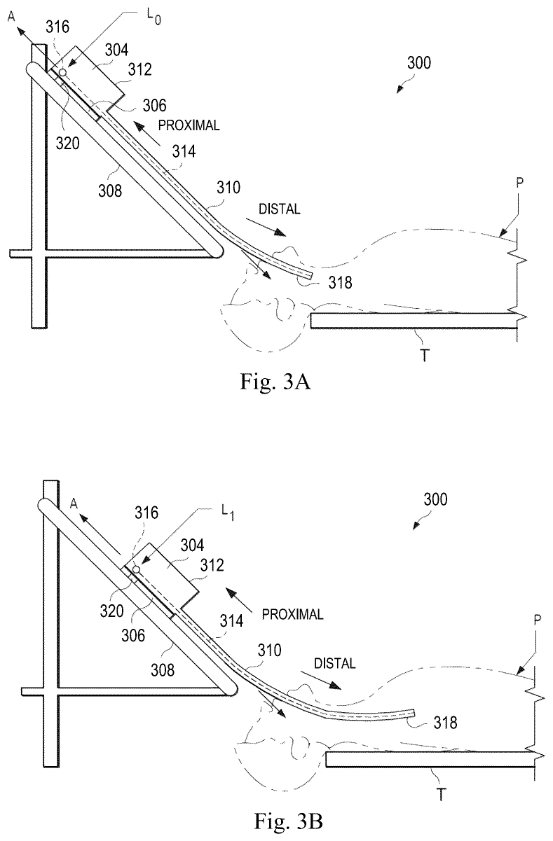

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

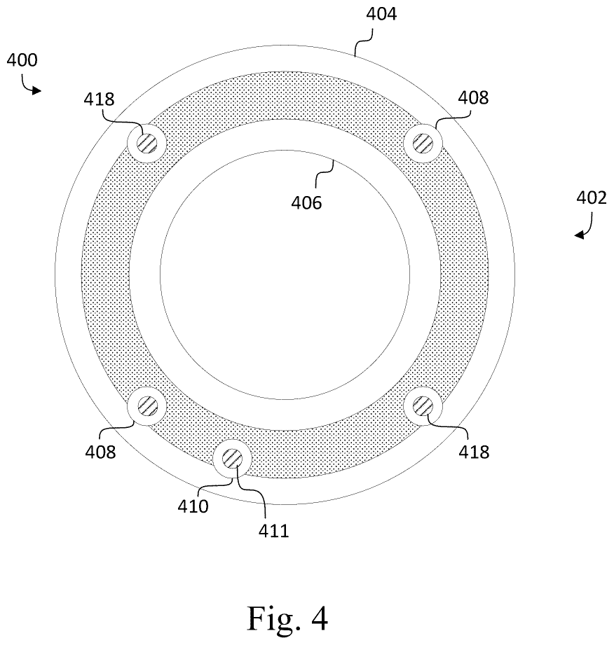

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

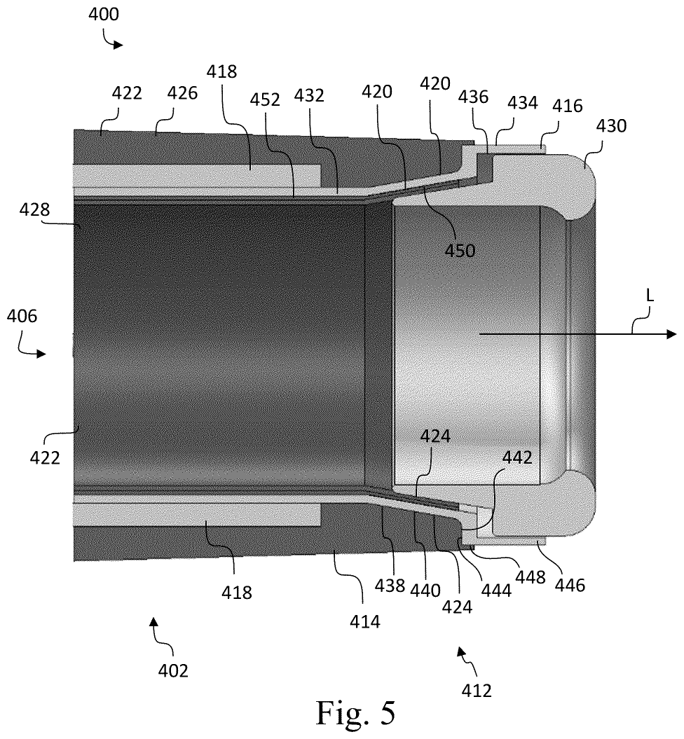

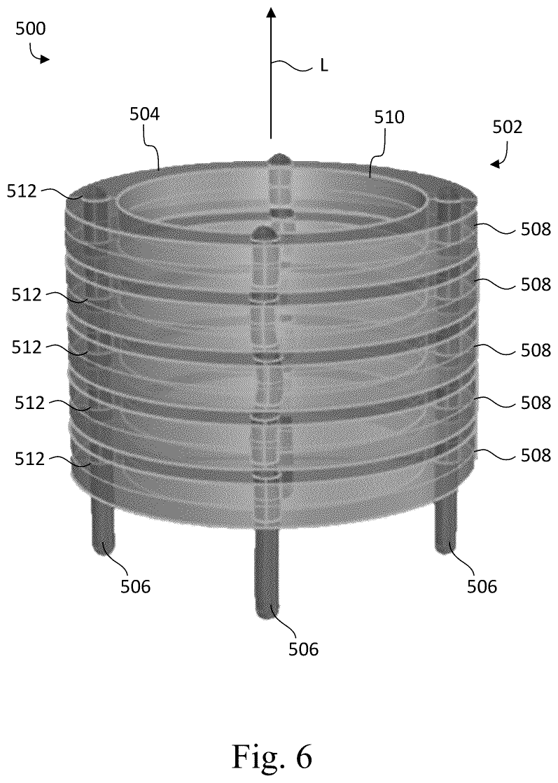

FIGS. 4 and 5 are simplified diagrams of a flexible elongate device 400 according to some embodiments. According to some embodiments consistent with FIGS. 1-3, the flexible elongate device 400 may correspond to the elongate device 202 of the medical instrument system 200.

As shown in the simplified cross-sectional diagram of FIG. 4, the flexible elongate device 400 can include a flexible body 402 with a flexible wall 404 having a thickness extending from an inner surface to an outer surface. A main lumen 406 can extend through the flexible body 402 and can provide a delivery channel for a medical tool, such as an endoscope, biopsy needle, endobronchial ultrasound (EBUS) probe, ablation tool, electroporation tool, chemical delivery tool, and/or the like, to be inserted through the flexible body 402. According to some embodiments, the flexible elongate device 400 can further include one or more control element lumens 408 arranged in the flexible wall around the main lumen 406 and/or one or more sensor lumens 410. The sensor lumens 410 can be utilized for any suitable component 411, including (e.g., shape) sensors, electrical wires, fibers, and/or the like.

As shown in FIG. 5, the flexible body 402 can include an articulable body portion 412 in a distal section 414 thereof. The articulable body portion 412 includes a control structure 416 attached with one or more control elements 418 that control articulation of the articulable body portion 412. In some examples, the control elements 418 can include pull wires, tendons, push rods, and/or the like. The control elements 418 can extend within the element lumens 408 shown in FIG. 4 or in any other structure in the proximal, intermediate, and the distal 414 sections of the flexible body 402 to connect to the control structure 416 by any suitable method. For example, the control elements 418 can connect to the control structure 416 by welding to the exterior of the control structure 416 (as shown in FIG. 5) or by extending within apertures of the control structure 416 as discussed in greater detail below.

In accordance with the disclosure herein, the articulable body portion 412 includes a seal 420 formed between a polymer material 422 and a sealing surface 424 of the control structure 416. The control structure 416 can be at least partially made of a metallic material, such as stainless steel or the like, which defines the sealing surface 424. The sealing surface 424 is functionalized by a primer to ensure that the seal 420 is satisfactorily formed. The molecules of the primer react with the metallic sealing surface 424 and become covalently bonded with the metallic material of the control structure 416 to functionalize the sealing surface 424. The seal 420 is beneficial for reusable devices to prevent reprocessing chemicals or biomatter from becoming trapped between the polymer material 422 and the control structure 416, ensuring that the devices are in a suitable condition for reuse.

To perform the functionalization, the primer is applied to the sealing surface 424. After application, the primer can then be cured on the sealing surface 424. The primer can be cured at room temperature, or by applying at least one of heat or pressure. For example, the primer can be cured in an oven with an elevated temperature. As a result of this process, the metallic scaling surface 424 is covered by the reacted primer molecules. The polymer material 422 is then disposed onto the sealing surface 424, and the reacted primer molecules bond thereto, which creates the seal 420 therebetween. A shrink tube or bonder, such as a radial bonder or an iris bonder, or a combination of both shrink tube and bonder, can be applied over the polymer to apply uniform compression pressure to enhance interaction between the polymer material and the functionalized sealing surface/primer and produce uniform lamination.

In one approach, the surface 424 is cleaned and then dipped in the primer for a predetermined amount of time. A solvent is then applied to the surface 424 to remove any excess primer. Next, the primer is allowed to dry, which causes the primer molecules to bond to the surface 424. After the surface 424 is prepared, the polymer material 422 is laminated onto the surface 424. In one example, a bonder device is used to apply heat and pressure to localize the heat onto the polymer material 422 aligned with the surface 424. The application of heat and pressure aids with the bonding of the polymer material 422 to the primer molecules bonded to the surface 424.

In the illustrated example, the distal section 414 of the flexible body 402 includes the control structure 416, which can be an annular control ring as shown, an outer jacket 426 at least partially defining an exterior surface of the articulable body portion 412, and an inner liner 428 at least partially defining the main lumen 406 of the flexible elongate device 400. In some examples, the distal section 414 can further include a tip ring 430. As shown, the inner liner 428 is sandwiched between the trip ring 430 and the control structure 416. Additionally, the tip ring 430 can include an inwardly protruding lip 430a. The reduced inner diameter provided by the lip 430a relative to an inner diameter of the main lumen 406 acts as a stopper for tools inserted through the main lumen 406, such as a vision probe. A similar stopper structure can alternatively be incorporated into the control structure 416. The control structure 416 extends along a longitudinal axis L and can be defined by a tubular body 432 including an exterior surface 434 and an interior surface 436. The outer jacket 426 and inner liner 428 can be made from any suitable polymer material, including polyurethane of various durometers, e.g., high durometer polyurethanes, or a stiffer material having a hardness or durometer higher than polyurethane. Suitable examples can include nylon, polyetheretherketone (PEEK), and so forth. The outer jacket 426 and/or inner liner 428 can be deposited onto the control structure 416 by any suitable method, including lamination.

The scaling surface 424 can be defined by at least part of the exterior surface 434 of the control structure body 432 and the polymer material 422 can be the outer jacket 426. Pursuant this this, the body 432 of the control structure 416 includes a proximal section 438 defining an exterior surface portion 440 that generally faces outwardly and extends generally parallel with or at an acute angle with respect to the longitudinal axis L. The sealing surface 424 of this embodiment includes at least a portion of the exterior surface portion 440 of the control structure proximal section 438, such that the seal 420 seals the outer jacket 426 to the exterior surface portion 440 of the control structure 416.

In some examples, the control structure body 432 can further include an intermediate section 442 that extends radially outwardly in a direction transverse, e.g., orthogonal, to the longitudinal axis L of the control structure 416 to expand the outer diameter of the control structure 416. The intermediate section 442 defines a second, lateral exterior surface portion 444 of the exterior surface 434. The second exterior surface portion 444 is disposed adjacent to and extends laterally away from the first portion 440. With this configuration, the outer jacket 426 can be disposed on the control structure 416 to extend along the first exterior surface portion 440 and abut the second exterior surface portion 444 of the intermediate section 442. If desired, the scaling surface 424 can include a portion or all of the second exterior surface portion 444, such that the outer jacket 426 is sealed to multiple sections 438, 442 of the control structure 416.

In further examples, the body 432 can further include a distal section 446 that extends away from the intermediate section 442 in a direction generally parallel with the longitudinal axis L of the control structure 416. The distal section 446 defines a third exterior surface portion 448 that generally faces outwardly and runs generally parallel with the longitudinal axis L. With this configuration, the outer jacket 426 can be disposed on the control structure 416 to extend along the first exterior surface portion 440 of the proximal section 438, along the second exterior surface portion 444 of the intermediate section 442, and at least partially along the third exterior surface portion 448. If desired, the sealing surface 424 can include a portion or all of the third exterior surface portion 448, such that the outer jacket 426 is sealed to three sections 438, 442, 446 of the control structure 416.

In additional examples, the sealing surface 424 can be defined by at least part of the interior surface 436 of the control structure body 432 and the polymer material 422 can be the inner liner 428. Pursuant this this, the proximal section 438 of the body 432 also defines an interior surface portion 450 that generally faces inwardly and extends generally parallel with or at an outwardly acute angle with respect to the longitudinal axis L. The sealing surface 424 of this embodiment is defined by at least part of the interior surface portion 450 of the control structure proximal section 438, such that the seal 420 seals the inner liner 428 to the interior surface portion 450 of the control structure 416. The control structure 416 may include one or more sealing surfaces. Each sealing surface may be functionalized by a primer to facilitate bonding with polymer material. In some examples, one or more portions of the control structure 416 that is not a sealing surface may also be functionalized by a primer.

In any of the above embodiments, the primer can be a phosphonate, such as a phosphonic acid, an alkyl phosphonate, or a phosphonate ester derivative. With this embodiment, the functionalized metallic material of the sealing surface 424 includes a self-assembled monolayer of phosphonates comprised of a phosphonic acid and a carbon-based molecule with a functional tail group. Alternatively, the primer can be a silane, such as aminosilane. With this alternative embodiment, the functionalized metallic material of the sealing surface 424 includes organofunctional alkoxysilane molecules. After reaction with the metallic material of the sealing surface 424, the reacted primer is covalently bonded with the sealing surface 424 of the control structure 416. The primer can be applied to the metallic sealing surface 424 by any suitable method. For example, the primer can be by one or more of: swabbing, dip coating, pipetting, spraying, and plasma deposition.

In any of the above forms, the sealing surface 424 can be pre-treated before application of the primer to ensure better application of the primer and functionalizing of the sealing surface 424. For example, the sealing surface 424 can be treated by grit-blasting or via a plasma surface treatment. A plasma surface treatment can be used to clean the sealing surface 424, as well as deposit the primer onto the sealing surface 424, if desired.

In any of the above forms, a tie layer 452 can be deposited on the sealing surface 424 prior to functionalizing the sealing surface 424 with the primer. For example, an oxide layer can be deposited on the sealing surface 424 to be disposed between the polymer material 422 and the control structure 416. Any suitable oxide layer can be utilized, including, for example, a silicon oxide layer or an aluminum oxide layer. In these examples, the primer can then be utilized to functionalize the oxide layer.

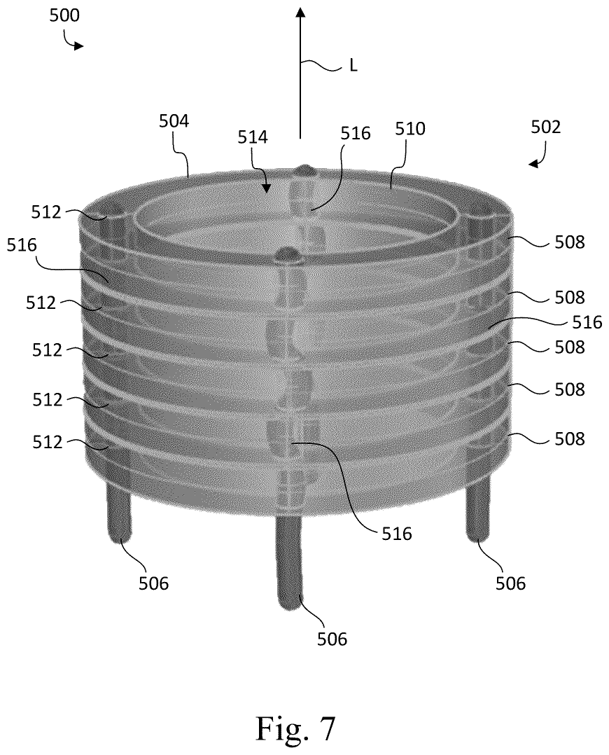

FIGS. 6-9 are simplified diagrams of a flexible elongate device 500 having a control structure assembly 502 according to some embodiments. According to some embodiments consistent with FIGS. 1-5, the flexible elongate device 500 may correspond to the elongate device 202 of the medical instrument system 200 or the flexible elongate device 400.

As shown in the simplified diagrams of FIGS. 6-8, the control structure assembly 502 includes a control structure 504 and one or more pull wires 506. The control structure 504 extends along a longitudinal axis L and is divided into a plurality of control segments 508 that are stacked along the longitudinal axis L. The control structure 504 can include any desired number of control segments 508, including 2, 3, 4 or more, 5 or more, 6 or more, and so forth. In the illustrated example of the control structure 504 having a ring shape, each of the control segments 508 also has a corresponding ring shape. It will be understood that the control segments 508 are shown in FIGS. 6 and 7 as partially transparent for the sake of clarity to show the control structure assembly 502 and should not be interpreted to limit the material of the control segments 508 in any regard. Furthermore, the control structure and control segments may include other types of shapes. Although the control structure 504, and control segments 508 thereof, are described herein as having a ring shape, it will be understood that other configurations, such as those without a central lumen or aperture, can alternatively be utilized. For example, the control structure assembly 502 it suitable for other steerable tools, e.g., a heart mapping catheter, or non-medical steerable tools, e.g., a borescope or plumber's scope.

Each of the control segments 508 define a tool channel aperture 510 and one or more pull wire apertures 512 depending on a desired degree of articulation for the flexible elongate device 500. The tool channel apertures 510 of the control segments 508 are coaxially aligned to at least partially form a tool channel 514 of the flexible elongate device 500. For example, as shown in the aligned tool channel apertures 510 when the control segments 508 are stacked define a cylindrical interior surface of the control structure 504 forming the tool channel 514.

The control structure assembly 502 can include any desired number of pull wires 506. For example, the assembly 502 can include one pull wire 506, two pull wires 506 (e.g., for articulation along a pitch or yaw axis), three pull wires 506, four pull wires 506 (e.g., for articulation along pitch and yaw axes), and so forth. Each control segment 508 can include a pull wire aperture 512 for each pull wire 506 distributed around the tool channel aperture 510 thereof. For example, the pull wire apertures 512 can be evenly spaced around the tool channel aperture 510. With two pull wires 506, the two pull wire apertures 512 can be disposed on opposite sides of the control segment 508. With four pull wires 506, the four pull wire apertures 512 can be disposed at generally 90-degree increments, for example.

“Corresponding” pull wire apertures 512, as used herein, refer to the pull wire apertures 512 that receive the same pull wire 506. As shown in the FIGS. 6-8, corresponding pull wire apertures 512 of adjacent control segments 508 are offset relative to one another. “Offset,” as used herein, means that corresponding pull wire apertures 512 of adjacent control segments 508 have some overlap, but are not entirely aligned. In alternative implementations, the pull wire apertures 512 may be entirely misaligned, such that they have no overlap, if the control segments 508 are sufficiently deformable to allow the pull wires 506 to impress into the body of the control segments 508 when the control segments 508 are compressed together, as discussed below.

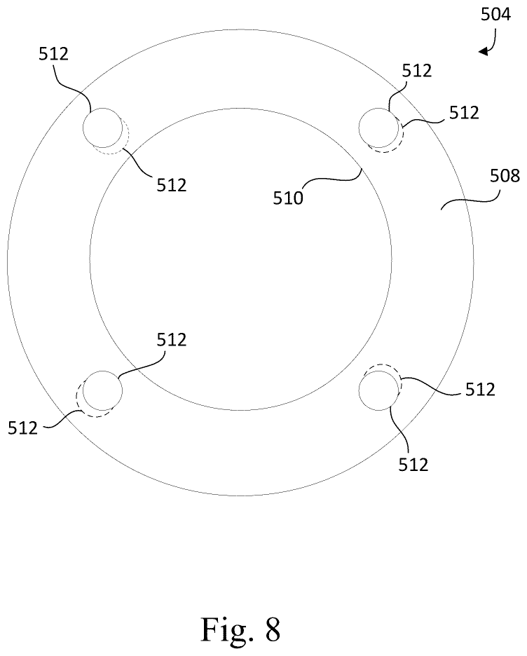

Corresponding pull wire apertures 512 of adjacent control segments 508 may be offset in a direction orthogonal to the longitudinal axis L of the control structure 504. For example, the corresponding pull wire apertures 512 can be offset in a circumferential and/or a radial direction. FIG. 8 illustrates a top control segment 508 shown in solid lines and a bottom control segment 508 shown in broken lines where the pull wire apertures 512 thereof are offset relative to the pull wire apertures 512 of the top control segment 508. For example, pull wire apertures 512 having radial offsets, e.g., inward and outward offsets, are shown on a left side of FIG. 8. In another example, pull wire apertures 512 having circumferential offsets, e.g., clockwise and counterclockwise offsets, are shown on a right side of FIG. 8. It will be understood that the offsets shown in FIG. 8 are for explanation and clarification. Any combination of radial and/or circumferential offsets can be utilized within the scope of disclosure provided herein. In some examples, a circumferential offset can be between 1 degree and 5 degrees, or about 3 degrees. In some examples, a radial offset can be between about 0.01-0.06 mm, or about 0.03 mm. In some examples, some of the corresponding pull wire apertures 512 of adjacent control segments 508 may be aligned or fully offset. The offsets may provide a reduced dimension opening between the apertures 512 of adjacent control segments 508. The pull wires 506 may have a dimension, e.g., diameter, greater than the reduced dimension opening. For example, the dimension of the pull wires 506 may be 5% larger, 2% larger, ±1% larger, 0.5% larger, 0.2% larger, 0.1% larger, or 0.05% larger. With this configuration, the pull wire 506 is compressed/pinched between the control segments 508 when the control segments 508 are forced together.

Due to the offset of the pull wire apertures 512, a secured portion 516 of the pull wire 506 extending through the corresponding pull wire apertures 512 of the control segments 508 has a non-linear shape. The non-linear shape of the secured portion 516 advantageously creates a capstan effect and utilizes friction as a result of the pull wire 506 bending and engaging the surfaces of the control segments 508, which holds the pull wire 506 within the control structure 504 without the need for knotting, welding, crimping, soldering, and so forth. In some examples, the pull wire 506 is not attached to the control structure 504 via knotting, welding, crimping, or soldering, thereby simplifying the manufacturing process.

With embodiments having two or more pull wires 506, the non-linear shapes of the secured portions 516 of at least a pair of adjacent pull wires 506 can be mirror images of one another to resist rotation of the control segments relative to one another. For example, and for case of explanation, with a circumferential offset, a first pull wire 506 can include, in sequential control segments 508, a first clockwise circumferential offset, a second counterclockwise circumferential offset, a third clockwise circumferential offset, and a fourth counterclockwise circumferential offset. Simultaneously, a second pull wire 506 adjacent to the first pull wire 506 can include, in the same sequential control segments 508, a first counterclockwise circumferential offset, a second clockwise circumferential offset, a third counterclockwise circumferential offset, and a fourth clockwise circumferential offset. With this mirrored configuration, the non-linear shapes of the pull wires 506 will impart counteracting forces on the control segments 508. Similarly, an assembly 502 utilizing radial offsets can vary radially inward and radially outward offsets to give the secure portions of the pull wires 506 mirror configurations. It will be understood that assemblies 502 utilizing circumferential and radial offsets can have similarly mirrored configurations.

In another example, using a clock as an orientation reference, a first control segment 508 can have pull wire apertures 512 at 12 o'clock, 3 o'clock, 6 o'clock, and 9 o'clock and a second control segment 508 can have pull wire apertures 512 at 1 o'clock, 2 o'clock, 7 o'clock, and 8 o'clock positions. It will be understood that the relative offsets in this example are for illustration and should not be understood to limit smaller offsets. Further, a third control segment 508 can include pull wire apertures 512 at 12 o'clock, 3 o'clock, 6 o'clock, and 9 o'clock, while a fourth control segment 508 can have pull wire apertures 512 at 11 o'clock, 4 o'clock, 5 o'clock, and 10 o'clock. If desired, a fifth control segment can have pull wire apertures 512 at 12 o'clock, 3 o'clock, 6 o'clock, and 9 o'clock.

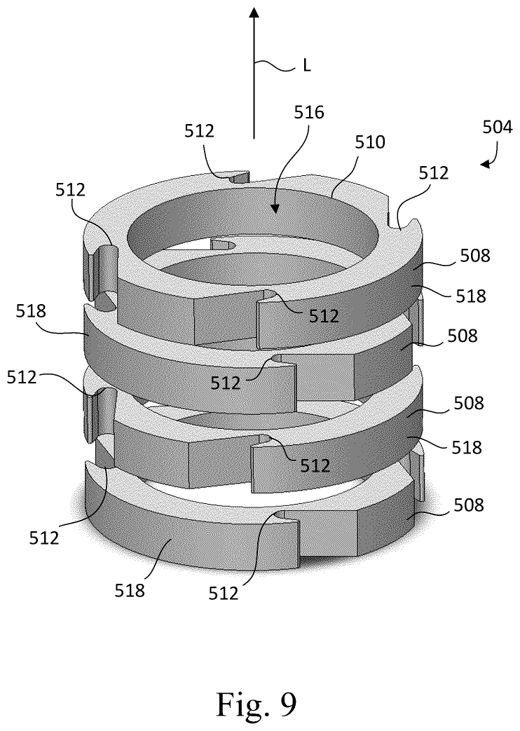

The pull wire apertures 512 can take any suitable form. For example, the pull wire apertures 512 can be a throughbore as shown in FIGS. 6-8. In another example as shown in FIG. 9, the pull wire apertures 512 can be hooks open through an outer surface 518 of the control segments 508. The hook configuration allows the pull wires 506 to be side loaded into the pull wire apertures 512, as compared to being threaded through the throughbore of the embodiment shown in FIGS. 6 and 7. In some examples, the hook pull wire apertures 512 of adjacent control segments have different orientations. The orientation of the hook pull wire apertures 512 can be switched from one control segment 508 to the next to capture the pull wire 506 therein, such that the wire apertures 512 extend in a sequential clockwise, counterclockwise pattern between adjacent control segments 508. The hook configuration can reduce assembly times and can be advantageous for pull wire materials that may catch while being threaded through a throughbore, such as multifilament or fibrous material.

In some instances, it may be desirable to skew one or more pull wire aperture in a direction away from a path of travel of the pull wire 506. For example, the control structure 504 can include a sequentially/incrementally increased skew of the pull wire apertures 512 relative to the longitudinal axis L of the control structure 504 going from a lowest or no skew in a proximal control segment 508 to a highest skew in a distal control segment 508. Gradually increasing a skew of the pull wire apertures 512 provides a greater hold on the pull wires 506 distally within the control structure 504, while also minimizing the of kinking the pull wire 506 at a proximal end of the control structure 504.

In one approach, the pull wire(s) 506 can be inserted through or loaded into the pull wire apertures 512 with the control segments 508 spaced from one another. Thereafter, the control segments 508 can be compressed together into a compressed state, which traps the secured portion(s) of the pull wire(s) 506 in the non-linear shape. Thereafter, the control segments 508 can be secured in the compressed state. The control segments 508 can be secured together by any suitable method, including welding or shrink tubing, for example. After assembly, any excess pull wire 506 extending distally past the distal control segment 508 can be removed.

The control structure 504 and pull wire(s) 506 can be made from any suitable material. In some examples, the materials of the control structure 504 and pull wire(s) 506 can have a similar strength. For example, the control structure 504 and pull wire(s) 506 can both be stainless steel. In other examples, the pull wire(s) can be a polymer, e.g., a liquid crystal polymer, a fiber, e.g., an Aramid fiber, or a metal cable, e.g., a multi-filament metal cable.

It will be understood that the control structure assembly 502 described with reference to FIGS. 6-9, including the control structure 504 thereof, can be utilized in combination the flexible elongate device 400 described with reference to FIGS. 4 and 5. Similarly, the control structure 504 described with reference to FIGS. 6-9 can be functionalized with a primer to form a seal with a polymer material as described with reference to FIGS. 4 and 5. For example, a metallic scaling surface of an inner and/or outer surface of the control structure 504 can be functionalized with a primer to be bonded with an outer jacket and/or inner liner.

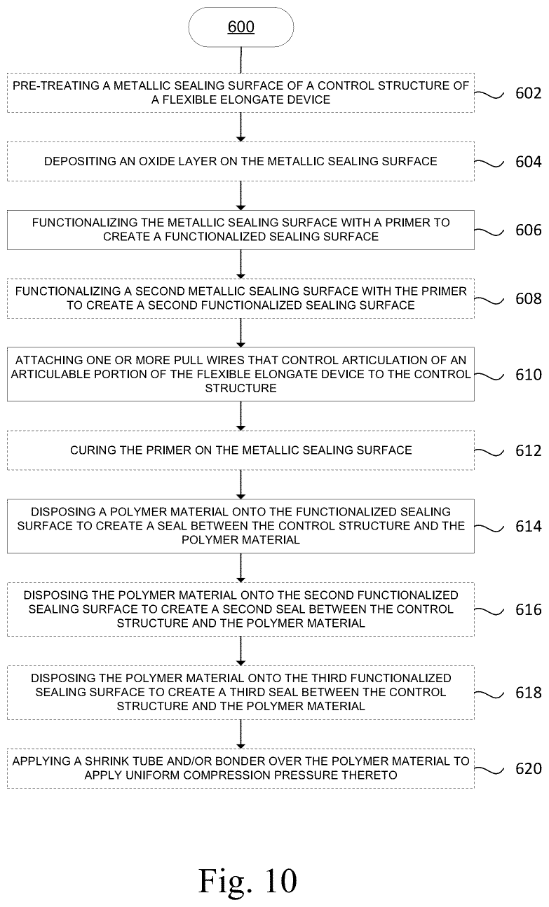

FIG. 10 illustrates a method 600 for assembly of a flexible elongate device (e.g., the flexible elongate device 202, 400, 500) according to some embodiments. The method 600 is illustrated as a set of operations or processes 602 through 616. Not all of the illustrated processes may be performed in all embodiments of the method 600. Additionally, one or more processes that are not expressly illustrated in FIG. 10 may be included before, after, in between, or as part of the processes 602 through 616. Processes may also be performed in different orders. In some embodiments, one or more of the processes 602 through 616 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 602 through 616 may be performed by a controller.

At process 602, a metallic surface of a sealing surface (e.g., sealing surface 424) of a control structure (e.g., control structure 416) is pre-treated, such as by grit blasting or plasma surface treatment. At process 604, an oxide layer is deposited on the metallic sealing surface. At process 606, the metallic sealing surface is functionalized with a primer to create a functionalized sealing surface. In some examples, process 606 can include functionalizing the metallic scaling surface with a phosphonate, covalently bonding a reacted primer with the metallic sealing surface, and/or applying the primer onto the metallic sealing surface by one of: swabbing, dip coating, pipetting, spraying, and plasma deposition. In other examples, process 606 can include functionalizing the metallic sealing surface with a silane, covalently bonding a reacted primer with the metallic sealing surface, and/or applying the primer onto the metallic sealing surface by one of: swabbing, dip coating, pipetting, spraying, and plasma deposition.

In some examples, the control structure can include a tubular body (e.g., tubular body 432) that defines the metallic sealing surface on an exterior surface of a proximal section (e.g., proximal section 438) thereof and a second metallic sealing surface on an exterior surface of an intermediate section (e.g., intermediate section 442) of the tubular body extending radially outwardly of the proximal section. In these examples, at process 608, the second metallic scaling surface is functionalized with the primer to create a second functionalized sealing surface. In further examples, the tubular body of the control structure can define a third metallic sealing surface on an exterior surface of a distal section (e.g., distal section 446) of the tubular body extending longitudinally away from the intermediate section. In these examples, at process 608, the third metallic sealing surface is functionalized with the primer to create a third functionalized scaling surface.

At process 610, one or more pull wires (e.g., pull wires 418, 506) that control articulation of an articulable portion of the flexible elongate device are attached to the control structure. In some examples, process 610 can include threading the one or more pull wires through pull wire apertures (e.g., pull wire apertures 512) defined in each of a plurality of control segments (e.g., control segments 508) forming the control structure stacked along a longitudinal axis thereof. In these examples, the pull wire apertures of adjacent control segments can be offset relative to one another in a direction orthogonal to the longitudinal axis of the control structure such that a secured portion of the pull wire extending through the pull wire apertures has a non-linear shape.

At process 612, the primer can be cured on the metallic sealing surface of the control structure. Curing can be achieved at room temperature or by applying at least one of: heat or pressure. At process 614, a polymer material is disposed onto the functionalized sealing surface to create a seal between the control structure and the polymer material. In some examples, process 612 can include laminating the polymer material onto the functionalized sealing surface, disposing an outer jacket (e.g., outer jacket 426) defining an exterior surface of the articulable body portion onto an exterior surface of the control structure, and/or disposing an inner liner (e.g., inner liner 428) defining an interior channel of the articulable body portion onto an interior surface of the control structure. At process 616, the polymer material is disposed onto the second functionalized sealing surface to create a second seal between the control structure and the polymer material. At process 618, the polymer material is disposed onto the third functionalized sealing surface to create a third seal between the control structure and the polymer material.

In optional process 620, after the polymer material is laminated onto the functionalized sealing surface, e.g., first, second, and/or third functionalized sealing surface, one or both of a shrink tube or a bonder, e.g., an iris or radial bonder, may be applied over the polymer material to apply uniform compression pressure thereto, which may enhance interaction between the polymer material and the functionalized sealing surface.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

The terms “generally,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A flexible elongate device, comprising:
an articulable body portion;
a control structure attached with one or more pull wires that control articulation of the articulable body portion, the control structure comprising a metallic material defining a sealing surface functionalized by a primer; and
a polymer material disposed onto the sealing surface of the control structure to create a seal between the control structure and polymer material.

2. The flexible elongate device of claim 1, wherein the primer comprises a phosphonate or a silane.

3. The flexible elongate device of claim 1, wherein the primer comprises a reacted primer covalently bonded with the sealing surface of the control structure.

4. The flexible elongate device of claim 1, wherein the polymer material comprises an outer jacket defining an exterior surface of the articulable body portion; and the sealing surface comprises an exterior surface portion of the control structure.

5. The flexible elongate device of claim 4, further comprising:
an inner liner defining an interior channel of the articulable body portion; and
a tip ring coupled to a distal end of the articulable body portion, the inner liner including a portion captured between the tip ring and the control structure.

6. The flexible elongate device of claim 1, wherein the polymer material comprises an inner liner defining an interior channel of the articulable body portion; and the sealing surface comprises an interior surface portion of the control structure.

7. The flexible elongate device of claim 1, wherein the control structure comprises a tubular body having a proximal section with an exterior surface defining the sealing surface and an intermediate section defining a lateral surface disposed adjacent to and extending away from the sealing surface, and the polymer material is disposed onto the control structure to abut the lateral surface.

8. The flexible elongate device of claim 7, wherein the intermediate section defines a second sealing surface functionalized by the primer on the lateral surface, and the polymer material is disposed on the second sealing surface such that the seal extends to the second sealing surface.

9. The flexible elongate device of claim 8, wherein the tubular body of the control structure further comprises a distal section extending longitudinally from the intermediate section and defining a third sealing surface functionalized by the primer, and the polymer material is disposed on the third sealing surface such that the seal extends to the third sealing surface.

10. The flexible elongate device of claim 1, further comprising a tie layer disposed between the polymer material and the control structure.

11. The flexible elongate device of claim 10, wherein the tie layer comprises an oxide layer.

12. The flexible elongate device of claim 1, wherein the polymer material is one of: a high durometer polyurethane or a material having a hardness higher than polyurethane.

13. The flexible elongate device of claim 1, wherein the control structure comprises a plurality of control segments stacked along a longitudinal axis of the control structure, and at least one of the one or more pull wires comprises a secured portion having a non-linear shape extending through the plurality of control segments.

14. The flexible elongate device of claim 13, wherein each of the control segments define a tool channel aperture and one or more pull wire apertures to receive individual ones of the one or more pull wires therethrough, the tool channel apertures of the plurality of control segments being coaxially aligned to at least partially form a tool channel of the flexible elongate device and the pull wire apertures of adjacent control segments for the at least one of the one or more pull wires being offset relative to one another in a direction orthogonal to the longitudinal axis of the control structure to form the secured portion having the non-linear shape.

15. A method for assembly of a flexible elongate device, the method comprising:
functionalizing a metallic sealing surface of a control structure with a primer to create a functionalized sealing surface;
attaching one or more pull wires that control articulation of an articulable body portion of the flexible elongate device to the control structure; and
disposing a polymer material onto the functionalized sealing surface to create a seal between the control structure and the polymer material.

16. The method of claim 15, wherein functionalizing the metallic sealing surface of the control structure of the primer comprises functionalizing the metallic sealing surface of the control structure with a phosphonate or a silane.

17. The method of claim 15, wherein functionalizing the metallic sealing surface of the control structure comprises covalently bonding a reacted primer with the metallic sealing surface of the control structure.

18. The method of claim 17, wherein covalently bonding the reacted primer with the metallic sealing surface of the control structure comprises:
applying the primer to the sealing surface; and
allowing the primer to dry on the sealing surface.

19. The method of claim 15, wherein disposing the polymer material onto the functionalized sealing surface comprises laminating the polymer material onto the functionalized sealing surface.

20. The method of claim 15, wherein disposing the polymer material onto the functionalized sealing surface comprises at least one of:
disposing an outer jacket defining an exterior surface of the articulable body portion onto an exterior surface of the control structure; or
disposing an inner liner defining an interior channel of the articulable body portion onto an interior surface of the control structure.

21. (canceled)

22. The method of claim 15, wherein the control structure comprises a tubular body defining the metallic sealing surface on an exterior surface of a proximal section thereof and a second metallic sealing surface on an exterior surface of an intermediate section of the tubular body extending radially outwardly of the proximal section, the method further comprising:
functionalizing the second metallic sealing surface to create a second functionalized sealing surface; and
disposing the polymer material onto the second functionalized sealing surface to create a second seal between the control structure and the polymer material.

23. The method of claim 22, wherein the tubular body of the control structure further comprises a third metallic sealing surface on an exterior surface of a distal section of the tubular body extending longitudinally away from the intermediate section, the method further comprising:
functionalizing the third metallic sealing surface to create a third functionalized sealing surface; and
disposing the polymer material onto the third functionalized sealing surface to create a third seal between the control structure and the polymer material.

24. The method of claim 15, further comprising applying at least one of a shrink tube or a bonder over the polymer material disposed onto the control structure to apply uniform compression pressure thereto.

25. The method of claim 15, further comprising applying at least one of heat or pressure to the polymer material aligned with the sealing surface with a bonder device.

26. The method of claim 15, further comprising pre-treating the metallic sealing surface by grit blasting or plasma surface treatment.

27. The method of claim 15, wherein functionalizing the metallic sealing surface of the control structure with the primer comprises applying the primer onto the metallic sealing surface by one of: swabbing, dip coating, pipetting, spraying, and plasma deposition.

28. The method of claim 15, further comprising curing the primer on the metallic sealing surface of the control structure.

29. The method of claim 15, further comprising depositing an oxide layer on the metallic sealing surface prior to functionalizing the metallic sealing surface of the control structure with the primer.

30. The method of claim 15, wherein attaching the one or more pull wires that control articulation of the articulable body portion of the flexible elongate device to the control structure comprises threading the one or more pull wires through pull wire apertures defined in each of a plurality of control segments forming the control structure stacked along a longitudinal axis thereof, wherein the pull wire apertures of adjacent control segments are offset relative to one another in a direction orthogonal to the longitudinal axis of the control structure such that a secured portion of the pull wire extending through the pull wire apertures has a non-linear shape.

31.-46. (canceled)
